(19) United States
(12) Patent Application Publication
(10) Pub. No.: US 2022/0143285 A1
(43) Pub. Date: May 12, 2022

(54) DIALYSIS SYSTEM AND METHODS

(71) Applicant: OUTSET MEDICAL, INC., San Jose, CA (US)

(72) Inventors: Dean HU, San Leandro, CA (US); Logan RIVAS, Aptos, CA (US)

(21) Appl. No.: 17/522,778

(22) Filed: Nov. 9, 2021

Related U.S. Application Data
(60) Provisional application No. 63/111,360, filed on Nov. 9, 2020.

(51) Int. Cl. A61M 1/16

(52) U.S. Cl. CPC A61M 1/1621; A61M 2205/3334; A61M 1/1668; A61M 1/1615

(57) ABSTRACT

Dialysis systems and methods are described which can include a number of features. The dialysis systems described can be to provide dialysis therapy to a patient in the comfort of their own home. The dialysis system can be configured to prepare purified water from a tap water source in real-time that is used for creating a dialysis fluid solution. The dialysis systems described also include features that make it easy for a patient to self-administer therapy.

### CROSS REFERENCE TO RELATED APPLICATIONS

This application claims the benefit of priority of U.S. Provisional Application No. 63/111,360, filed Nov. 9, 2020, herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure generally relates to dialysis systems. More specifically, this disclosure relates to systems and methods for creating dialysis fluid in real-time during dialysis treatment.

### BACKGROUND

There are, at present, hundreds of thousands of patients in the United States with end-stage renal disease. Most of those require dialysis to survive. Many patients receive dialysis treatment at a dialysis center, which can place a demanding, restrictive and tiring schedule on a patient. Patients who receive in-center dialysis typically must travel to the center at least three times a week and sit in a chair for 3 to 4 hours each time while toxins and excess fluids are filtered from their blood. After the treatment, the patient must wait for the needle site to stop bleeding and blood pressure to return to normal, which requires even more time taken away from other, more fulfilling activities in their daily lives. Moreover, in-center patients must follow an uncompromising schedule as a typical center treats three to five shifts of patients in the course of a day. As a result, many people who dialyze three times a week complain of feeling exhausted for at least a few hours after a session.

Many dialysis systems on the market require significant input and attention from technicians prior to, during, and after the dialysis therapy. Before therapy, the technicians are often required to manually install patient blood tubing sets onto the dialysis system, connect the tubing sets to the patient, and to the dialyzer, and manually prime the tubing sets to remove air from the tubing set before therapy. During therapy, the technicians are typically required to monitor venous pressure and fluid levels, and administer boluses of saline and/or heparin to the patient. After therapy, the technicians are often required to return blood in the tubing set to the patient and drain the dialysis system. The inefficiencies of most dialysis systems and the need for significant technician involvement in the process make it even more difficult for patients to receive dialysis therapy away from large treatment centers.

Given the demanding nature of in-center dialysis, many patients have turned to home dialysis as an option. Home dialysis provides the patient with scheduling flexibility as it permits the patient to choose treatment times to fit other activities, such as going to work or caring for a family member. Unfortunately, current dialysis systems are generally unsuitable for use in a patient's home. One reason for this is that current systems are too large and bulky to fit within a typical home. Current dialysis systems are also energy-inefficient in that they use large amounts of energy to heat large amounts of water for proper use. Although some home dialysis systems are available, they generally are difficult to set up and use. As a result, most dialysis treatments for chronic patients are performed at dialysis centers.

Hemodialysis is also performed in the acute hospital setting, either for current dialysis patients who have been hospitalized, or for patients suffering from acute kidney injury. In these care settings, typically a hospital room, water of sufficient purity to create dialysis fluid is not readily available. Therefore, hemodialysis machines in the acute setting rely on large quantities of pre-mixed dialysis fluid, which are typically provided in large bags and are cumbersome for staff to handle. Alternatively, hemodialysis machines may be connected to a portable RO (reverse osmosis) machine, or other similar water purification device. This introduces another independent piece of equipment that must be managed, transported and disinfected.

Dialysis fluids are required to have proper overall osmolar and specific electrolytic composition, in order to preserve blood cell structure and blood electrolyte content. Additionally, they are required to have high microbial purity to reduce risk of infection. The simplest way these can be provided is fully constituted, wherein they are mixed, packaged and sterilized at a facility, and shipped to their point of use (e.g., hospital, clinic or home). While this is straightforward, each dialysis treatment may require tens or even hundreds of liters of fluid, so this becomes logistically challenging and costly. Alternatively, dialysis fluids can be reduced to their base components (sodium chloride, dextrose, sodium bicarbonate, etc.), preferably in powdered form, and shipped to a treatment site. There, the components may be mixed in a batch process with purified water (typically from a reverse osmosis system) and filtered to produce the reconstituted dialysis fluid, or dialysis fluid concentrates, which can then be used placed in containers for use on individual dialysis machines. While this is more efficient from a shipping standpoint, it requires bulky equipment, and for scenarios where there is not high usage of dialysis fluid (such as a single patient home), much of the economies of scale are lost. Single-treatment batching systems are known, wherein purified water is mixed into a bag containing concentrated liquid. However these systems require many hours for batching, and are not suited for real-time, on-demand applications.

In another arrangement, dialysis fluid concentrates may be provided in pre-mixed liquid form, which are mixed on-line on a single dialysis machine together in real-time with a stream of purified water and filtered to produce the final dialysis fluid. For hemodialysis applications, a bicarbonate concentrate, containing aqueous sodium bicarbonate, and an acid concentrate, containing all the other components of dialysis fluid are provided separate. This is known as three-stream proportioning (acid, bicarbonate and water). The separation is necessary because the low pH of the acid concentrate is required to keep certain electrolytes such as calcium in solution, and when mixed with the bicarbonate, the pH increases. These two concentrates are typically provided in jugs, gallon sized or larger. While more suitable for low-use environments without mixing and/or distribution infrastructure, and more economical than shipping fully constituted dialysis fluid, this still has drawbacks. Lifting and manipulating gallon jugs may be challenging for physically limited patients at home. Even with the reduced volume relative to fully constituted dialysis fluid, shipping and storage of jugs is onerous.

To overcome these issues with three-stream proportioning, higher stream proportioning, such as four-stream proportioning, has been proposed and implemented in some machines. The technology has been around for several decades. In four stream proportioning, the acid concentrate is split into a dry sodium chloride which is dissolved similar to the sodium bicarbonate, and a smaller electrolyte liquid solution that contains the rest of the contents of the acid concentrate. This significantly reduces the amount and weight of supplies needed to conduct a treatment. A third volumetric pump can be introduced to pump the electrolyte stream. However, a final conductivity check may not be sufficient in this case to ensure proper mixing, because the contribution to conductivity from the electrolyte stream may be relatively small compared to the other streams. Its absence, or gross inaccuracy may not be detected by a single conductivity signal after mixing which is the summation of the contributions of the sodium chloride, bicarbonate and electrolyte. To overcome this limitation, another mixer and conductivity sensor may be placed in the system, which would mix the electrolyte stream with the water stream before the sodium chloride or bicarb. While this scheme is feasible, it adds a number of components, particularly ones which are potentially costly. Flow sensors may be used in place of the conductivity sensors, which are also costly.

The current state of the art in dialysis fluid preparation provides limited flexibility for modifying the dialysis fluid for the patient's needs, either before treatment, or during treatment in response to sensor biofeedback. For example, in the three-stream proportioning scheme described above, it is possible to increase or reduce either the acid concentrate flow, or bicarbonate concentrate flow, either as a setting prior to treatment, or during treatment. This allows adjustment of the bulk osmolarity of the solution (for example to encourage vascular refill to effectively remove excess fluid), as well as the total buffer content. However, since all of the individual minor electrolytes (calcium, potassium, etc.) are dissolved in bulk with the acid, these will change in lock-step with the acid concentrate flow rate. It is not possible to change the composition of minor electrolytes relative to each other, or the composition of sodium (the electrolyte that comprises the majority of the dialysis fluid's osmolarity). Often, particularly in the hospital setting, it may be necessary to change the concentration of potassium in a patients' dialysis fluid, even mid-treatment, in response to laboratory measurements. In this case, the treatment must be paused, the acid jug removed, and a jug of different acid concentration formulation that contains the proper potassium must be put in its placed. This is disruptive of the treatment workflow. Additionally, to account for the needs of different patients in terms of minor electrolyte concentrations, providers must stock many different variations of acid concentrate with different levels of calcium, potassium or other electrolytes. Typically, many different acid concentrates must be stocked with different levels of calcium, potassium or magnesium, or packets of electrolytes must be mixed into the acid to adjust it to the desired level. This setup can be prone to human error, and requires additional logistical burden of storing/tracking many different acid concentrate variations.

In the three-stream proportioning scheme, in addition to lifting, manipulating and opening the two jugs, the user must position the jugs individually, remove two connectors from their docking points on the machine, place the connectors into the jugs (sometimes with the additional step of attaching straws or wands), remove the two connectors from the jugs when treatment is complete, and then reattach the two connectors to their docks on the machine. Because of the high salt content of the fluids being handled, these connectors and their docks require frequent cleaning to remove the precipitated salts from minor drips, which can impair their function.

Therefore, there exists a need for a novel concentrate packaging and re-constitution scheme that is able to support real-time on-demand dialysis fluid creation without needing to wait for batching, provides less bulk and weight than liquefied jug concentrates to improve logistics and handling, provides the ability to adjust individual minor electrolytes independent of bulk osmolality and other minor electrolytes, and provides a user interface that requires fewer steps and less frequent cleaning.

### SUMMARY OF THE DISCLOSURE

A method of providing dialysis therapy is provided, comprising the steps of fluidly coupling a disposable container to a dialysis machine with a single connection interface, the disposable container including at least one compartment having a powdered dialysis fluid component therein and at least one compartment having a liquid dialysis fluid component therein, connecting a blood tubing set of the dialysis machine to a patient, initiating dialysis therapy, delivering a purified water into the at least one compartment having the powdered dialysis fluid component, generating dialysis fluid in real-time from the disposable container, and initiating dialysis therapy with the dialysis fluid.

In some embodiments, the method further comprises detecting a condition of the patient, and adjusting proportioning of the dialysis fluid in real-time.

In one embodiment, mounting the disposable container further comprises attaching a connector-side interface of the disposable container to a corresponding machine-side interface on the dialysis machine.

In another embodiment, detecting a condition further comprises measuring at least one parameter of the patient's health or the patient's blood.

In some examples, the condition comprises a state of electrolytes in the patient's blood.

In one embodiment, adjusting proportioning further comprises increasing a concentration of at least one dialysis fluid component from the disposable container.

In another embodiment, increasing a concentration of at least one dialysis fluid component further comprises increasing a proportion of fluid from the at least one compartment having the powdered dialysis fluid component.

In some embodiments, increasing a concentration of at least one dialysis fluid component further comprises increasing a proportion of fluid from the at least one compartment having the liquid dialysis fluid component.

In one example, the method further comprises decoupling and removing the disposable container from the dialysis machine, applying a cover to a machine-side connector of the single connection interface, and initiating a disinfect cycle through one or more flow paths of the dialysis machine and the machine-side connector.

A disposable container configured to facilitate real-time dialysis fluid production is provided, comprising at least one powder compartment configured to hold a powdered dialysis fluid component, at least one liquid compartment configured to hold a liquid dialysis fluid component, a connector interface configured to mate with a dialysis machine, at least one inlet flow path configured to deliver purified water from the dialysis machine through the connector interface to the at least one powder compartment, and a plurality of outlet flow paths configured to deliver dialysis fluid components from the at least one powder compartment and the at least one liquid compartment to the connector interface and the dialysis machine.

In some embodiments, the at least one powder compartment comprises a NaCl powder compartment and a NaHCO₃ powder compartment.

In another embodiment, the at least one liquid compartment comprises a C₆H₈O₇ liquid compartment, a C₆H₁₂O₆ liquid compartment, and a MgCl₂ liquid compartment.

In some examples, the disposable container further comprises a diffuser/filter disposed in the outlet flow paths between the at least one powder compartment and the connector interface.

In some examples, the connector interface further comprises a container-side connector interface configured to mate with a corresponding machine-side connector interface.

In other embodiments, the connector interface comprises at least one inlet flow channel and a plurality of outlet flow channels.

In one embodiment, the at least one inlet flow channel is fluidly coupled to a source of purified water.

In another embodiment, the at least one inlet flow channel is configured to deliver purified water to the at least one powder compartment.

In some embodiments, the machine-side connector interface further comprises a valve disposed in the at least one inlet flow channel, wherein the valve is configured to open when the container-side connector interface is connected to the machine-side connector interface.

In some examples, each of the plurality of outlet flow channels comprises a pump configured to deliver a dialysis fluid from the at least one powder compartment and the at least one liquid compartment to the dialysis machine.

In one embodiment, each of the plurality of outlet flow channels comprises a flow sensor configured to measure a flow rate of dialysis fluid from the at least one powder compartment and the at least one liquid compartment to the dialysis machine.

In one example, the at least one powder compartment and the at least one liquid compartment are large enough to generate enough dialysis fluid to support multiple dialysis treatments.

A dialysis machine is provided, comprising a connector interface disposed on or in the dialysis machine, the connector interface being configured to be coupled with a container including one or more sources of dialysis fluid, the connector interface being coupled to at least one purified water flow channel and a plurality of flow channels configured to receive the one or more sources of dialysis fluid when the container is coupled to the connector interface, and a rinsing cover disposed on the dialysis machine, the rinsing cover being configured to be moved into a rinsing configuration in which the rinsing cover forms a fluidic seal with the connector interface, wherein in the rinsing configuration purified water flows from the at least one purified water flow channel into a volume defined by the rinsing cover and the connector interface and further flows into the plurality of flow channels.

In some embodiments, in the rinsing configuration, a valve opening member of the rinsing cover is configured to open a valve in the at least one purified water flow channel.

In one example, the volume is further configured to receive a disinfecting pod.

In other examples, the rinsing cover further comprises one or more fluid channels configured to direct the purified water towards a periphery of the rinsing cover.

A method of generating dialysis fluid in real-time is provided, comprising operating at least one pump to generate a flow of purified water from a dialysis machine into at least one powder compartment of a disposable container, operating the at least one pump to generate a flow of a first liquid dialysis fluid component from the at least one powder compartment into the dialysis machine, operating the at least one pump to generate a flow of a second liquid dialysis fluid component from at least one liquid compartment of the disposable container into the dialysis machine, and proportioning, in the dialysis machine, the first liquid dialysis fluid component with the second liquid dialysis fluid component.

A flow circuit configured for real-time dialysis fluid production is provided, comprising at least one powder compartment configured to hold a powdered dialysis fluid component, at least one liquid compartment configured to hold a liquid dialysis fluid component, a source of purified water fluidly coupled to a purified water channel, a plurality of outlet flow paths configured to deliver liquid dialysis fluid components from the at least one powder compartment and the at least one liquid compartment to the connector interface and the dialysis machine, a valve disposed in each of the plurality of outlet flow paths and the purified water channel, each of the valves fluidly connected to an output flow path, an electronic controller configured to sequentially activate the valves to allow a first bolus of a dialysis fluid from at least one powder compartment or at least one liquid compartment to flow into the output flow path followed by a second bolus of purified water to flow from the purified water channel into the output flow path.

In some embodiments, the electronic controller activates only one of the valves to be open at any given time.

In other embodiments, the flow circuit includes a pump coupled to the output flow path downstream of the valves.

In one embodiment, the electronic controller is configured to synchronize activation of the valves with a rotational cycle of the pump.

In another embodiment, the electronic controller is configured to modulate a cycle time of the valves to adjust a concentration of the dialysis fluid from at least one powder compartment or at least one liquid compartment into the output flow path.

In one example, the flow circuit includes first and second conductivity sensors disposed in the output flow path downstream of the valves.

In other embodiments, the first and second conductivity sensors are configured to measure a conductivity of the dialysis fluid from at least one powder compartment or at least one liquid compartment in the output flow path to determine a volume of the first bolus.

In some examples, determining the volume further comprises identifying a series of peaks and troughs in the measured conductivity and quantifying an area under a curve of each of the series of peaks.

In other embodiments, the first conductivity sensor is optimized for a first flow rate and the second conductivity sensor is optimized for a second flow rate.

In one example, the first conductivity sensor has a flow channel diameter smaller than a flow channel diameter of the second conductivity sensor.

In another embodiment, the first conductivity sensor has a smaller sense size than that of the second conductivity sensor.

In some examples, the electronic controller is further configured to infer a composition of the dialysis fluid based on activation timing of the valves, the conductivity measurements, and a flow rate of the dialysis fluid.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows one embodiment of a dialysis system.

FIG. 2 illustrates one embodiment of a water purification system of the dialysis system.

FIG. 3 illustrates one embodiment of a dialysis delivery system of the dialysis system.

FIGS. 4A-4B illustrate various embodiments of a disposable container configured to mate to a dialysis machine to generate dialysis fluid in real-time.

FIGS. 5A-5D illustrate embodiments of a connector of a disposable container.

FIG. 6 is another embodiment of a disposable container configured to mate to a dialysis machine to generate dialysis fluid in real-time.

FIGS. 7A-7B illustrate one embodiment of a hinged cover that facilitates disinfecting of flow paths after removal of a disposable container.

FIG. 8A is one embodiment of a flow circuit for preparing dialysis fluid in real-time.

FIG. 8B is another embodiment of a flow circuit for preparing dialysis fluid in real-time

FIGS. 8C-8H illustrate methods and embodiments for determining a fluid stream composition using one or more conductivity sensors and valve sequences in a fluid circuit.

FIGS. 9A-9F illustrate some examples of a dialysis machine including a connector that can generate dialysis fluid from a disposable container or from conventional liquid jugs.

FIG. 10 is a flowchart for a method of providing dialysis therapy to a patient.

### DETAILED DESCRIPTION

This disclosure describes systems, devices, and methods related to dialysis therapy, including a dialysis system that is simple to use and includes automated features that eliminate or reduce the need for technician involvement during dialysis therapy. In some embodiments, the dialysis system can be a home dialysis system. Embodiments of the dialysis system can include various features that automate and improve the performance, efficiency, and safety of dialysis therapy.

In some embodiments, a dialysis system is described that can provide acute and chronic dialysis therapy to users. The system can include a water purification system configured to prepare water for use in dialysis therapy in real-time using available water sources, and a dialysis delivery system configured to prepare the dialysis fluid for dialysis therapy. The dialysis system can include a disposable cartridge and tubing set for connecting to the user during dialysis therapy to retrieve and deliver blood from the user.

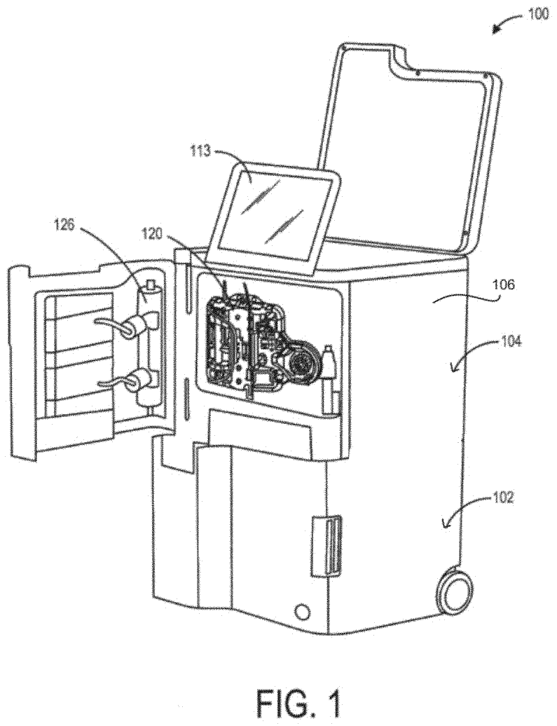

FIG. 1 illustrates one embodiment of a dialysis system 100 configured to provide dialysis treatment to a user in either a clinical or non-clinical setting, such as the user's home. The dialysis system 100 can comprise a water purification system 102 and a dialysis delivery system 104 disposed within a housing 106. The water purification system 102 can be configured to purify a water source in real-time for dialysis therapy. For example, the water purification system can be connected to a residential water source (e.g., tap water) and prepare purified water in real-time. The purified water can then be used for dialysis therapy (e.g., with the dialysis delivery system) without the need to heat and cool large batched quantities of water typically associated with water purification methodologies.

Dialysis system 100 can also include a cartridge 120 which can be removably coupled to the housing 106 of the system. The cartridge can include a patient tubing set attached to an organizer. The cartridge and tubing set, which can be sterile, disposable, one-time use components, are configured to connect to the dialysis system prior to therapy. This connection correctly aligns corresponding components between the cartridge, tubing set, and dialysis system prior to dialysis therapy. For example, the tubing set is automatically associated with one or more pumps (e.g., peristaltic pumps), clamps and sensors for drawing and pumping the user's blood through the tubing set when the cartridge is coupled to the dialysis system. The tubing set can also be associated with a saline source of the dialysis system for automated priming and air removal prior to therapy. In some embodiments, the cartridge and tubing set can be connected to a dialyzer 126 of the dialysis system. In other embodiments, the cartridge and tubing set can include a built-in dialyzer that is pre-attached to the tubing set. A user or patient can interact with the dialysis system via a user interface 113 including a display.

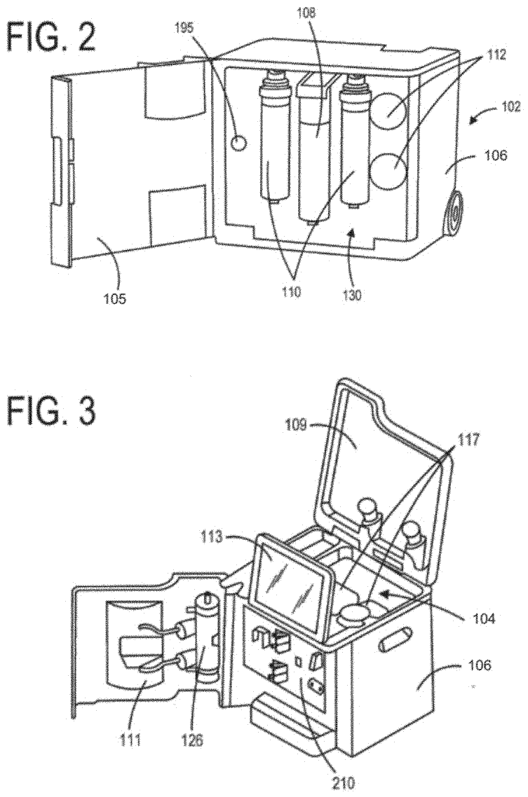

FIGS. 2-3 illustrate the water purification system 102 and the dialysis delivery system 104, respectively, of one embodiment of the dialysis system 100. The two systems are illustrated and described separately for ease of explanation, but it should be understood that both systems can be included in a single housing 106 of the dialysis system. FIG. 2 illustrates one embodiment of the water purification system 102 contained within housing 106 that can include a front door 105 (shown in the open position). The front door 105 can provide access to features associated with the water purification system such as one or more filters, including sediment filter(s) 108, carbon filter(s) 110, and reverse osmosis (RO) filter(s) 112. The filters can be configured to assist in purifying water from a water source (such as tap water) in fluid communication with the water purification system 102. The system can optionally include a chlorine sample port 195 to provide samples of the fluid for measuring chlorine content.

In FIG. 3, the dialysis delivery system 104 contained within housing 106 can include an upper lid 109 and front door 111, both shown in the open position. The upper lid 109 can open to allow access to various features of the dialysis system, such as user interface 113 (e.g., a computing device including an electronic controller and a display such as a touch screen) and dialysis fluid containers 117. Front door 111 can open and close to allow access to front panel 210, which can include a variety of features configured to interact with cartridge 120 and its associated tubing set, including alignment and attachment features configured to couple the cartridge 120 to the dialysis system 100. Dialyzer 126 can be mounted in front door 111 or on the front panel, and can include lines or ports connecting the dialyzer to the prepared dialysis fluid, dialysis fluid concentrate, liquid concentrate, etc., as well as to the tubing set of the cartridge. In one implementation, described in more detail below, the dialysis machine and/or dialyzer can include lines or ports configured to connect to a disposable reservoir that includes a plurality of powdered and/or liquid compartments for dialysis fluid preparation and delivery.

Described herein is are novel systems and methods for packaging all of the individual components necessary to constitute dialysis fluid in a single, multi-chamber, disposable reservoir container. This container can include chambers that contain both powdered components, as well as chambers that contain liquid components. The powdered components can be homogenous salts, for example, one compartment containing sodium chloride and another compartment containing sodium bicarbonate. These two components are preferentially provided in powder form because they comprise the majority of the components in dialysis fluid and provide the greatest volume and weight savings in powdered form compared to liquid form. The powder compartments, however, require flow paths to provide water for dissolution. At the point where the powder compartments join with the fluid flow path, a diffuser or filter can be placed to ensure only dissolved, saturated fluid is conveyed downstream.

The other compartments may be provided in concentrated liquid form because the overall mass content is lower, and therefore by providing them pre-dissolved, not much volume is used. Since no fluid paths are required for the liquid compartments, the design is simplified.

This disposable reservoir container may be fabricated from two flexible sheets that are welded together in a pattern to provide the empty volumes for the different compartments, and the flow paths. Other methods of manufacturing can be used, including thermoforming and various molding modalities. The flow paths from the various compartments can be arranged side-by-side to all meet at a connector component attached to the disposable reservoir container. The aggregated flow paths may further be disposed such that they extend from the component compartments as a flexible, tube or ribbon-like structure which can be easily maneuvered.

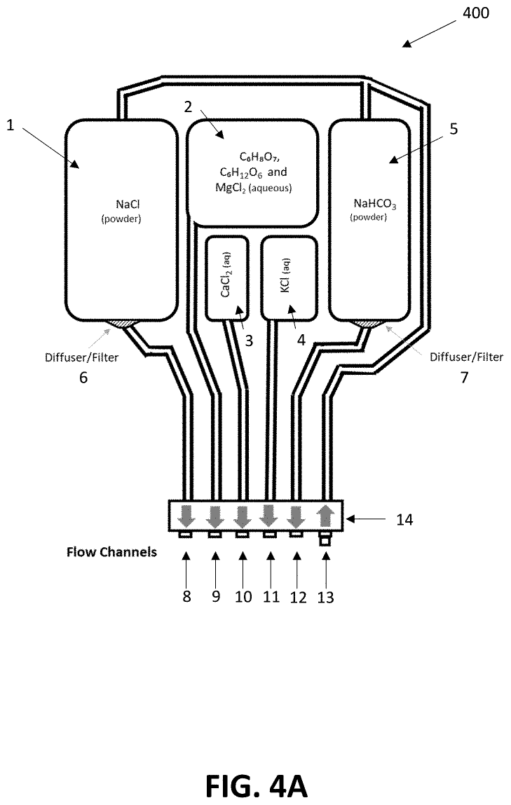

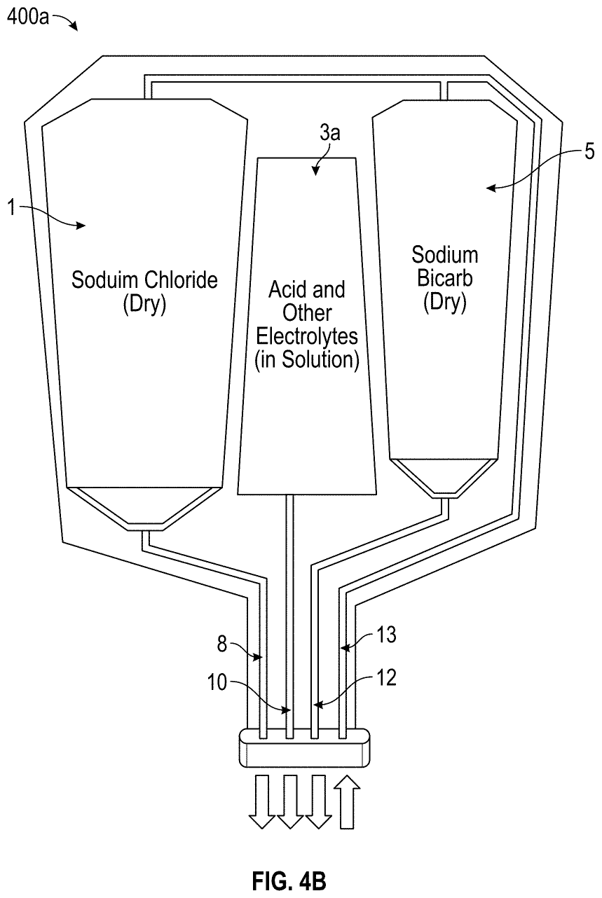

FIG. 4A is an illustration showing one embodiment of a disposable dialysis fluid preparation container 400. The container 400 can include a plurality of compartments that can have volumes sized and configured to provide a standard four-hour dialysis treatment at a dialysis fluid flow rate of 300 ml/min. Referring to FIG. 4A, the container 400 can include compartments 1-5, diffuser/filters 6-7, and flow channels 8-13. The compartments can be configured to house and store a combination of powdered and/or liquid concentrates necessary for the real-time production of dialysis fluid. Referring to FIG. 4A, the container 400 can include at least two powder compartments configured to house a powdered dialysis fluid component. For example, compartment 1 can be configured to hold sodium chloride (NaCl) powder and compartment 5 can be configured to hold Sodium Bicarb (NaHCO₃) powder. In one implementation, the compartments can be sized and configured to hold enough of each of the powders to provide enough dialysis fluid for a standard four-hour dialysis treatment. For example, a four-hour treatment may require 1,000 gms of NaCl and 500 gms of NaHCO₃, resulting in compartment volumes of 1000 mL and 500 mL for compartments 1 and 5, respectively. Referring still to FIG. 4A, it can be seen that the powder compartments 1 and 5 can further include diffusers/filters 6 and 7 to ensure only dissolved, saturated fluid is conveyed downstream. Additionally, flow channel 13 can provide a flow path for purified water to flow into compartments 1 and 5 to enable the production of dialysis fluid from the powder in those compartments. In some embodiments, the flow channel 13 can enter at or near a top of the compartments 1 and 5 to ensure that powder or concentrate within those compartments is fully wetted. In use, the purified water flows from fluid path 13 into compartments 1 and 5, where it mixes with the powder in each compartment, is filtered by filter/diffusers 6 and 7, and then flows back to the dialysis machine via flow channels 8 and 12.

In addition to the powder compartments 1 and 5, the container 400 of FIG. 4A can also include a plurality of liquid compartments, including compartments 2, 3, and 4. Since these compartments house chemical concentrates in liquid form, there is no need for a flow channel to provide purified water to these compartments. As shown in FIG. 4A, compartment 2 can include an aqueous mix of Citric Acid (C₆H₈O₇), Glucose (C₆H₁₂O₆), and Magnesium Chloride (MgCl₂). In one example, a four-hour treatment may require 7 gms of C₆H₈O₇ at 6% saturation, 45 gms of C₆H₁₂O₆ at 25% saturation, and 3.4 gms of MgCl₂ at 4% saturation, which would require compartment 2 to have a volume of at least 200 mL. Compartment 3 can include an aqueous solution of Calcium Chloride (CaCl₂). During a four-hour treatment, it is expected that 14 gms of CaCl₂ at a 35% saturation would be required, resulting in a compartment volume of 50 mL. Similarly, compartment 4 can include Potassium Chloride (KCl), and a four-hour treatment would require 14 gms of KCl at 77% saturation resulting in a compartment volume of 75 mL. Flow channels 9, 10, and 11 can be configured to deliver the solution from compartments 2, 3, and 4, respectively, to the dialysis system. In the embodiment of FIG. 4B, instead of a plurality of liquid compartments, a single liquid compartment 3a may be provided, into which all solutes other than sodium bicarbonate and sodium chloride are dissolved. While this embodiment would not allow for independent adjustment of each electrolyte, the fluidics are simplified while retaining the general size and logistics advantages.

In the embodiment of FIG. 4A, the disposable container includes 5 compartments, with two of the compartments comprising powdered sodium chloride and bicarb, and the other compartments comprising various aqueous solutions. This results in a 6-stream (or 6 flow channel) container: 5 flow channels for the respective compartments and a 6^{th} flow channel that provides purified water to the container. In other embodiments, 3 and 4 compartment containers are proposed. In a three compartment container 400a, as shown in FIG. 4B, Ca and K are not separated out into separate containers (containers 3 and 4 in FIG. 4A), but are instead combined into a small liquid container 3a which provides acid and other electrolytes in solution. Purified water is still fed to the container via flow channel 13, and proportioned solutions are fed back to the dialysis system with flow channels 8, 10, and 12. While this embodiment is simpler to manufacture and provides simpler hardware for the system to process, it still provides the logistical savings of powderizing the sodium chloride and bicarb. However, this embodiment does not allow for independent electrolyte adjustments or the ability to formulate any prescription from a single disposable.

In some embodiments, the disposable container is sized large enough to support multiple treatments, for example in an in-center or in-hospital scenario. In these scenarios, the reservoir can be kept mounted on the dialysis machine between treatments. Highly ionic, stagnant fluid in the flow paths, particularly pumps/valves is not desirable. While it would be possible to continue pumping forward to ensure movement, this would deplete the reservoir compartments. In these scenarios, the pumps and/or pump/valve assemblies can be programmed to pump backwards and forwards in an oscillatory manner, to prevent fluid stagnation and not cause reservoir depletion.

The container 400 can include a connector interface 14 with the dialysis machine in a multi-stream proportioning scheme. In some embodiments, the connector interface can plug or mate directly into with a corresponding connector on the dialysis machine. This connector interface provides multiple flow channels, including flow channel 13, as mentioned above, that provides purified water to the container in order to continuously dissolve the powdered components, as well as a plurality of flow channels 8-12 to receive flow from either the liquid compartments or dissolved powder compartments. The flow channel 13 may be connected to a spray chamber of the dialysis machine, which can be configured to de-aerate the purified water. Each of the other flow channels 8-12 can be configured to interface with one flow path, and can be individually sealed at the connector interface. In some embodiments, the connector interface comprises elastomer sealing members for each flow channel, such as tube-shaped structures, that interface with rigid mating structures on the corresponding connector of the dialysis machine. Additionally, the connectors can be rotationally asymmetrical to prevent mis-connection between the container and the dialysis machine.

The disposable container can, in combination with sensors on the dialysis machine, enable real-time proportioning adjustments and/or dialysis fluid flow rate changes. In one implementation, sensors in the dialysis machine can provide feedback, particularly for electrolyte levels in the patient's blood. This feedback can be displayed to the user, attending clinician, or sent to a nephrologist. Further, upon notification of the measured levels, a nurse and/or physician could adjust the level of electrolytes sent to the patient as needed. The measured feedback can also be used as a mode of control in the event that the flow sensor does not work as intended. By measuring the ionic concentration and cross referencing it against the flow meter, the two sensors can cross check one another as an added layer of safety.

Dialysis systems according to the present disclosure typically have a patient tubing set that includes, at a minimum, venous and arterial lines that connect to the patient, a dialyzer, and mechanisms for removing air from the tubing set such as a venous drip chamber. There are at least two locations where these sensors could be deployed within the patient tubing set of the dialysis system; directly facing the blood (e.g., in the blood line prior to the dialyzer), or on the used dialysis fluid line, after the dialyzer. The advantages of the blood-facing location are a more direct measurement of electrolytes in the patient. However, the drawbacks are the need to mount/unmount the sensors/substrate (i.e., blood cartridge) each treatment, and the additional cost added to the blood cartridge for sensing or interface components. Alternatively, the sensors may be positioned on the used dialysis fluid line, after the dialyzer. This is less cost-prohibitive, because the sensors and all interface components are built into the machine and are therefore reusable. However, the sensors and interface components must now be able to withstand disinfect cycles (heat, chemical), and also do not have as direct a measurement position.

Used dialysis fluid is new dialysis fluid (of a substantially known ionic composition) which has come into contact with blood through a semi-permeable membrane. Electrolyte exchange between the blood and the dialysis fluid will occur, and since electrolytes readily pass through the dialyzer membrane, it can be assumed that homogenous distribution of the electrolytes between blood and dialysis fluid will be reached once the used dialysis fluid exits the dialyzer. The concentration of homogeneity of any given electrolyte can be measured by the sensors on the used dialysis fluid line. Kinetically, the concentration of homogeneity, which is measured, can be calculated from the concentration in the new dialysis fluid (known), the dialysis fluid flow rate (known), the blood flow rate (known) and the concentration in the blood (unknown). The fluid removal rate can also be factored in, if present. From there, the concentration of the given electrolyte in the blood can be determined by inference.

While this relationship generally holds true, in a real-world application, it may be desirable to improve the sensitivity of these measurements by eliminating some of the contributing factors, even if they are nominally known. One novel aspect of this disclosure is to operate the system, during treatment, in a mode intended to provide the best measurement conditions of electrolyte composition for sensors disposed on the used dialysis fluid line. In a typical hemodialysis treatment, some flow of dialysis fluid is provided into the dialyzer. In other modes, no flow of dialysis fluid is provided to the dialyzer, but flow out of the dialyzer comprising ultrafiltrate is still enabled. This ultrafiltrate (when no incoming dialysis fluid flow is present) comprises the blood components that are able to pass through the filter—of which electrolytes are included. Additionally, since the electrolytes readily able to pass through the filter, their concentration in the ultrafiltrate should exactly match the concentration in blood. Therefore, by measuring the used dialysis fluid during a mode where no new dialysis fluid is supplied to the dialyzer, higher accuracy is obtained. The impact of dialysis fluid flow rate, blood flow rate, new dialysis fluid concentration all drop out of the equation. As an example, right at the beginning of treatment, new dialysis fluid flow may be paused, and ultrafiltration-only mode may be entered into for a brief period of time, in order to provide a good window of measurement. Based on these measurements, adjustments to the dialysis fluid electrolyte composition can be performed. When dialysis fluid flow is resumed, the inference method may be then used to detect any changes in concentration. Periodically, the machine may enter an ultrafiltration-only mode (which may be scheduled or on-demand) to perform a more accurate measurement, or provide a level of calibration to the inference-based method. This measurement window may further be combined with a step where the blood flow through the blood circuit is reversed, for example, to measure flow through the vascular access.

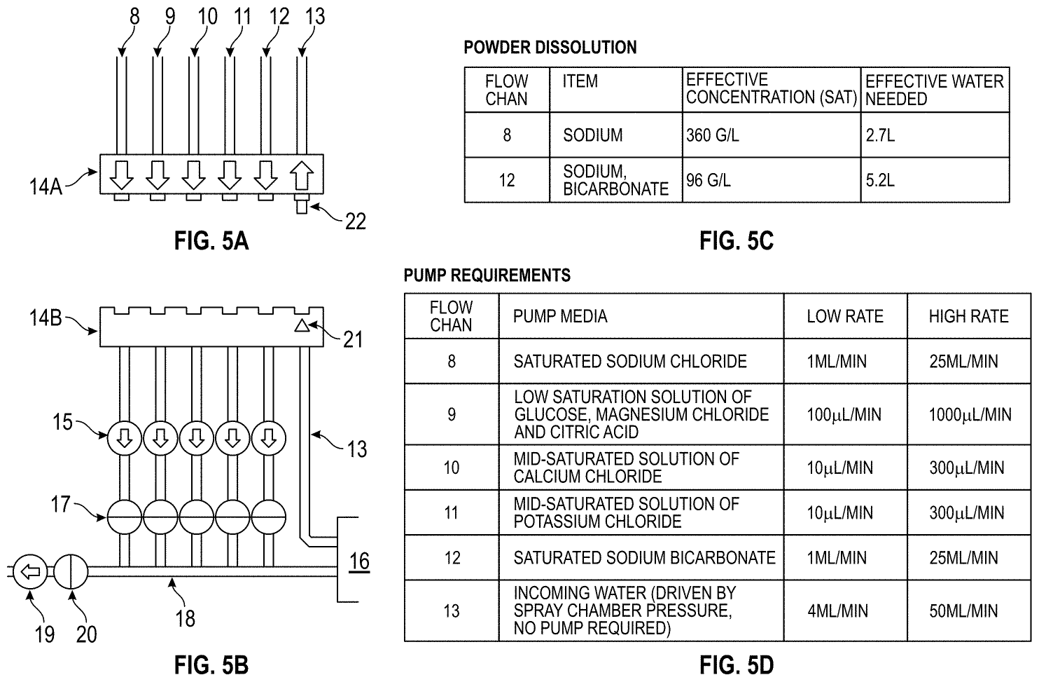

FIGS. 5A-5B illustrate one embodiment of a container-side 14A of the connector interface 14, and a dialysis machine-side 14B of the connector interface, respectively, including the flow channel 13 which provides a source of purified water to the container. These flow channels can be continuous from the individual compartments of the disposable container described above in FIG. 4A, and their attached flow paths. On the machine-side 14B, each of these flow channels may be connected a dedicated pump 15 and feedback sensor 17, for example a flow sensor. After metering the concentrate/solutions from the container by the pumps, each of the flow channels is joined to a primary purified water stream 18 emanating from the dialysis machine spray chamber 16, and connected to a dialysis fluid pump DP 19 for delivery for therapy. Optionally, an ion sensor array 20 can be included on this mixed stream to verify correct proportioning of all the electrolytes from the container. In one embodiment, the machine-side 14B can include a spring-loaded valve 21 which can be configured to be opened when mated with the container-side 14A. For example, the container-side 14A can include a valve opening member 22 configured to interface with the spring-loaded valve of the machine-side. When the container-side 14A is not connected to or mated with the machine-side 14B, the spring-loaded valve 21 can be in a closed configuration to prevent contaminants or other materials or liquids from entering the flow channel 13 and also prevent purified water from flowing out of the dialysis machine. However, when the container-side 14A is connected to or mated with the machine-side 14B, the valve opening member 22 engages spring-loaded valve 21 to allow purified water to flow from the dialysis machine into the flow channel 13 of the disposable container.

FIG. 5C is a table showing one embodiment of the effective concentration and water needed for proper dissolution during therapy and the corresponding flow channel through which that liquid concentrate travels through. FIG. 5D is a table showing one example of pump requirements for each flow channel to provide the appropriate amount of liquid form each of the compartments of the disposable container.

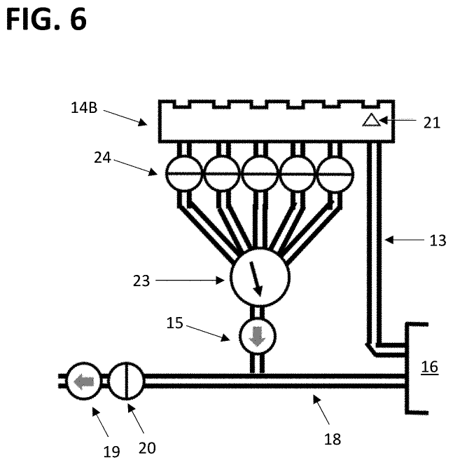

In an alternate embodiment of the connector on the machine-side 14B, as shown in FIG. 6, the flow channels from the container tie into one or more selector valves 23 on the machine-side connector 14B. One or more pumps 15 can be located downstream of each of the selector valves, and can operate at a nominally constant rate. The selector valve can sequentially connect each of the flow channels to the pump flow path, such that, by varying the duty cycle of the selector valve at each of its settings, the ratio of individual components may be altered. Due to the large differences in flow rates, multiple selector valve/pump configurations can be used, by grouping components with like flow rates together. In some embodiments, one or more flow sensors 24 can be associated with each of the flow channels prior to the selector valve 23.

Still referring to the machine-side connector, at least one of the channels of the connector comprises is an outlet channel (such as flow channel 13), configured to deliver purified water at low pressure to the disposable container reservoir, for continuous dissolution of the powder concentrates. This channel (denoted flow channel 13 above) may include a spring-loaded valve 21, which is closed when the container-side connector is not attached to the machine-side. The corresponding channel on the container-side connector can include a valve opening member, that when connected to the machine-side connector, opens the valve. This valve can be provided such that when the machine-side connector is not connected to the container-side connector, the flow of water is stopped (i.e., the valve is closed).

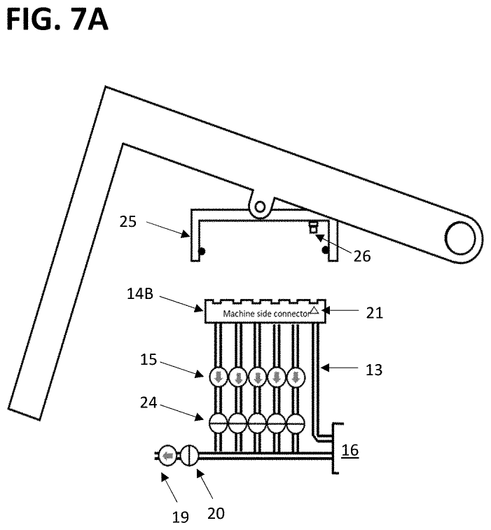

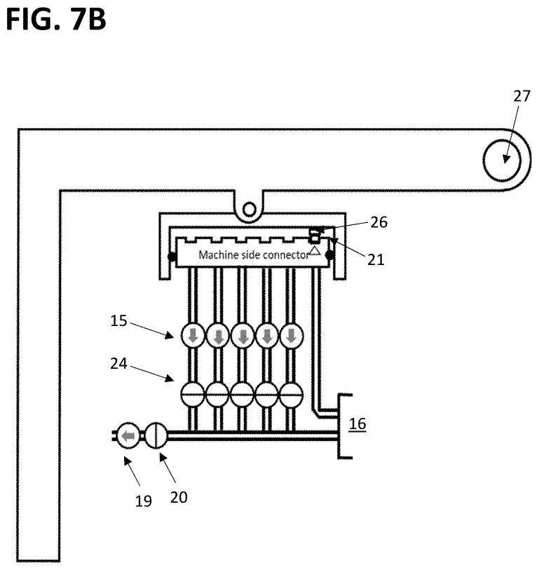

When the system is not in use, and specifically when the reservoir container is not connected, it is desirable to create continuous flow paths through all the flow channels on the machine side to allow the flow paths to be rinsed with purified water, or otherwise disinfected with either hot liquid or chemical disinfectant. In one embodiment, referring to FIGS. 7A-7B, a rinsing cover 25 is provided that covers the machine-side connector 14B when not in use. This rinsing cover can further comprise a valve opening member 26 that is positioned to open a corresponding spring-loaded valve 21 on the machine-side connector which connects to the purified water source of the machine-side connector. As should be understood, the valve opening member 26 performs the same function as the valve opening member 22 of the container-side 14A. The rinsing cover member is further configured to form a fluidic seal around the periphery of the machine-side connector, such that all individual inlet ports of the machine-side connector are left unoccluded and contained within an empty volume defined by the bottom surface of the rinsing cover member, the top surface of the machine-side connector and a peripheral seal formed by the rinsing cover member with the machine-side connector. Thus when the rinsing cover is in place, flow paths all emanating from the opened valve cover of the flow channel 13 to all of the other flow channels are created, which can be used for rinsing or disinfection.

Referring to FIG. 7B, the rinsing cover 25 is preferentially connected, either through a rotational joint 27, or rigidly, to a hinged panel that is on the dialysis machine. In one position, the hinged panel is closed, which positions the rinsing cover member sealably over the machine-side connector. If the rinsing cover member is coupled with a rotational joint to the hinged panel, a biasing torque on the rotational joint may be beneficial to force the rinsing cover member into a substantially horizontal position prior to contact, to overcome rotational misalignment. In another position, the hinged panel is open, which exposes the machine-side connector (as is shown in FIG. 7A). In this embodiment, the hinged panel can comprise a “L” shape, or two sections which are substantially perpendicular. The hinged panel may further comprise hooks or other features (located on the section of the hinged panel perpendicular to the section on which the rising cover lies) to allow mounting of the aforementioned disposable reservoir container. When mounted, the container-side connectors of the disposable reservoir container can be substantially in position to mate with the machine-side connector. In some embodiments, the mating and/or unmating of the two counterpart connectors can assisted by an electromechanical mechanism. For example, the user can place the container-side connector into a latched carrier in close proximity to the machine-side connector, which is then driven by a linear actuator into a position where the counterpart connector channels all mate together.

When closed, the hinged panel may further serve form a cosmetic cover for mechanisms on the machine, for example the connectors and holder areas for the dialyzer. This configuration allows the hinged cover to perform multiple functions, and thus save user steps: (1) expose for access the mechanisms that were covered, such as the dialyzer area; (2) unseal and expose for access the machine-side connector, and (3) provide a mounting point for the disposable reservoir. Similarly, when the treatment has completed, closing the hinged panel members also serves multiple functions.

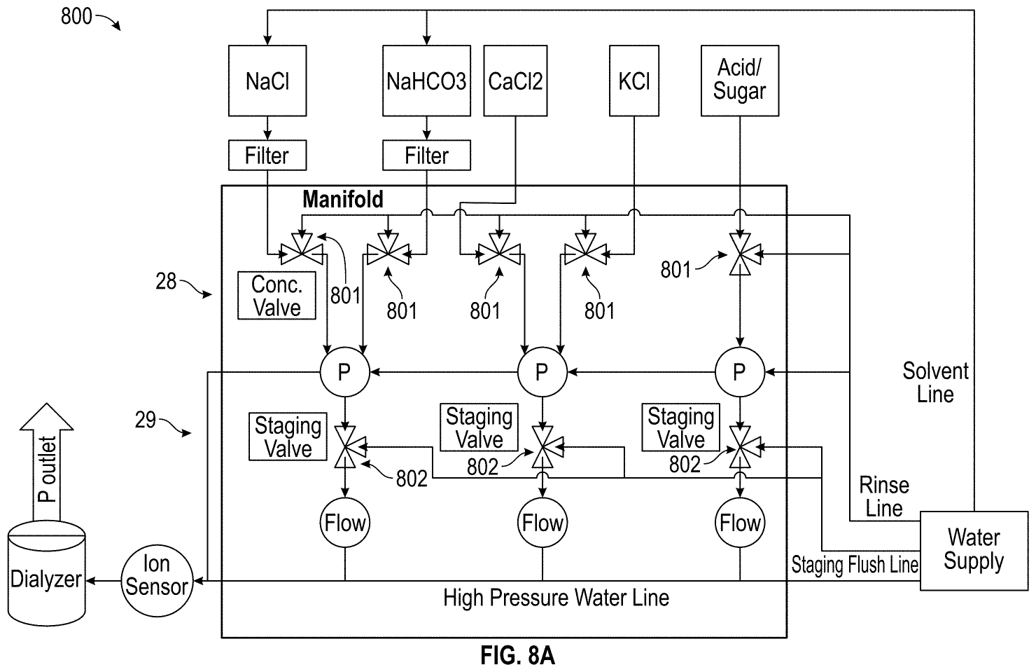

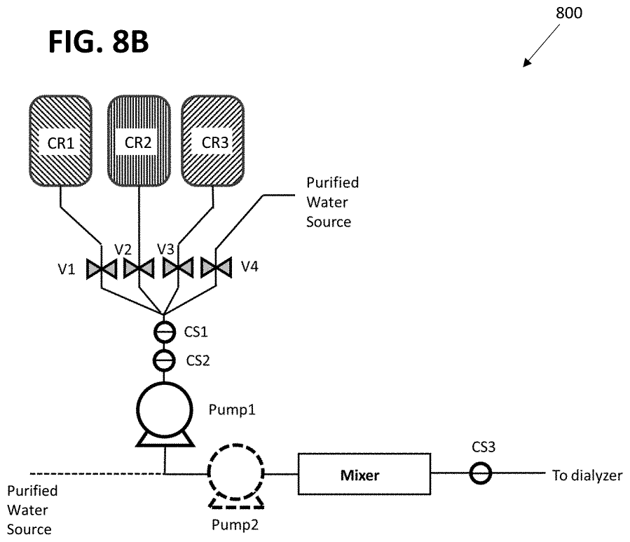

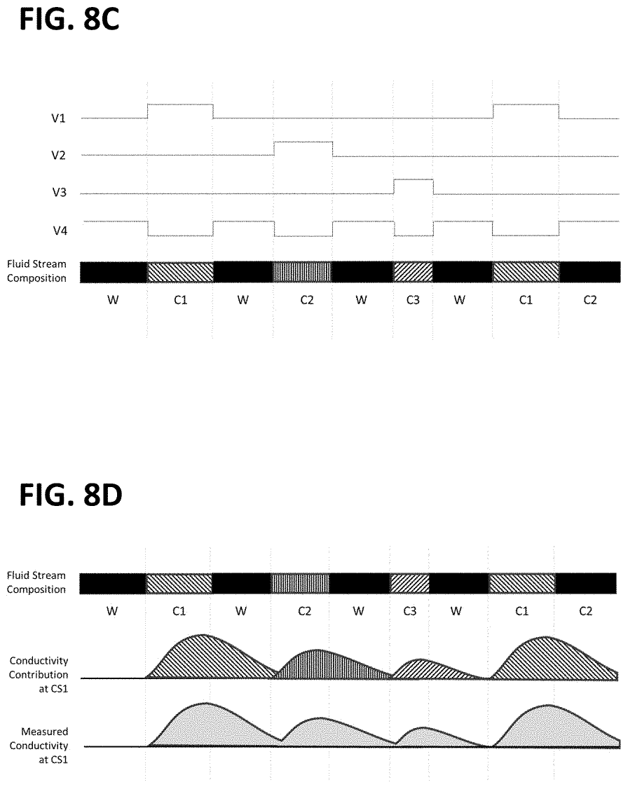

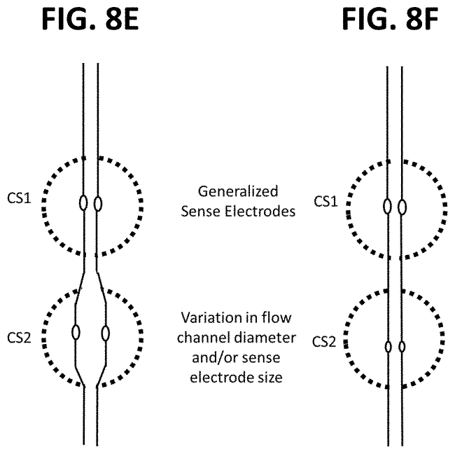

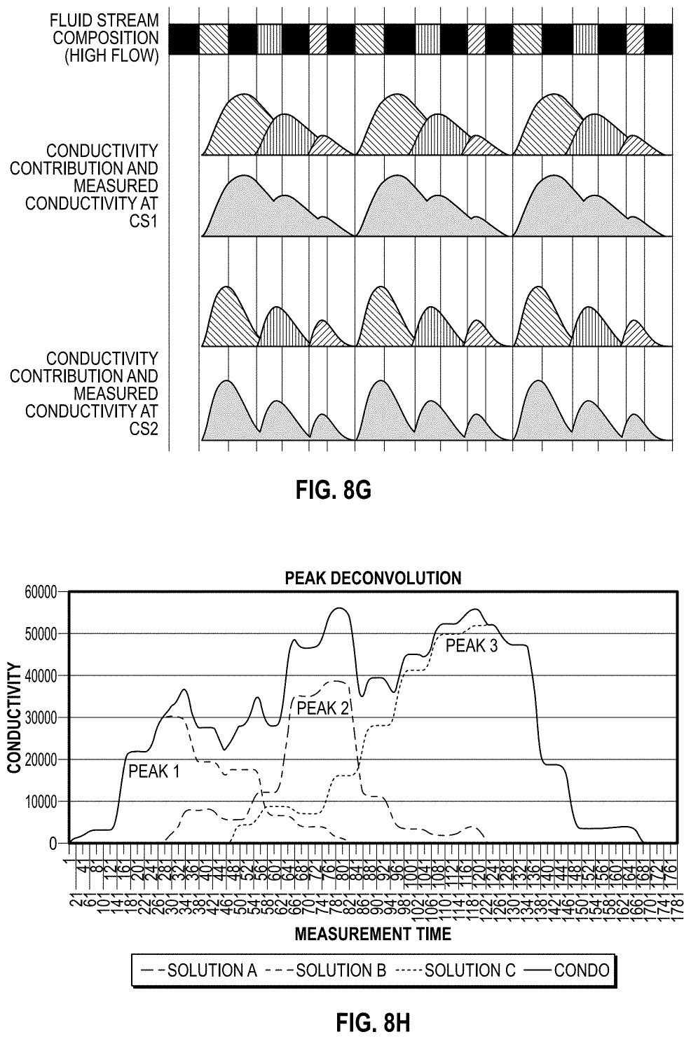

In another embodiment, referring to FIG. 8A, the dialysis machine can include a flow circuit 800 configured to employ an array of valves that are fluidically linked via shared manifold or multiple shared manifolds. The fluidically controlled circuit can enable the metering, timing and monitoring of fluid proportions from the compartments of the disposable container. An array of valves 801 and 802 can divide the circuit into a concentrate control inlet section 28 and a fluid staging section 29. Variable volume plumps P may be employed within the fluid handling circuit to allow for concentrates of similar volumes to share a common line. The valve array can be configured to switch between a desired solution for delivery to the main line. A single pump per concentrate solution could also be used keeping the concentrates isolated to their own respective fluid paths.

The concentrate control can allow for initial delivery of concentrate into the pump from the compartments of the disposable container. By selectively opening or closing the concentrate valves in the concentrate control inlet section 28 the concentrate can be delivered to the pump(s) or a second fluid can be delivered to the pump or back to the concentrate. This configuration would serve the utility of flushing the lines or providing a calibration fluid of a known physical property for the flow and ion sensors.

The fluid staging section 29 of the fluid handling circuit can be configured to serve the purpose of proving a metered volume of fluid ready for delivery upon the actuation of the staging valve(s) 802 and pump(s) P. An alternate embodiment could be a pump that drives multiple fluids and the timing of each staging valve would then control the dose. Issues arise in when uncontrolled or unspecified performance parameters from the manufacture would influence the volume being delivered. These may include, for example, the spring constant of the valve used, minute flow variations between pumps, changes in fluidic entry parameters such as a shoulder step size, flow diameter or surface finish. These variations could in turn create a flow restriction and unintentionally impact the volume of fluid delivered.

In another embodiment, the system would not make use of staging valves and the fluid would be delivered directly to the main line. In this embodiment, check valves could be used to prevent backflow from occurring as the pump aspirates from the concentrate line and delivers to the main flow line. Periodic flushing of the check valves may be required to prevent buildup from keeping the valves artificially open. The flow sensors could then be used as a mode of control to detect and trigger a system response in the event that uncontrolled flow is detected. Due to build up from the concentrates used, a routine flushing of the valves and pumps may be required to ensure long term reliability. A staging flush and rinse line can deliver a fluid to all components that allows the removal of any slow accumulation of the fluid during the life of the system. The cleaning procedure would be automated into the systems normal maintenance cycle.

FIG. 8B illustrates another embodiment of a flow circuit 800 for preparing dialysis fluid in real-time. Generally, the physical structure of the flow circuit comprises a plurality of concentrate reservoirs (CR1, CR2, CR3). These reservoirs may be provided in liquid form, or may alternatively house a powder that is continuously dissolved with purified water, or a combination thereof (e.g., one or more of the containers may include liquid and the other(s) powder). As described above, compartments or reservoirs containing a powder may be connected to a separate fluid line or channel that feeds purified water into the powder compartment for form a dialysis fluid. These reservoirs could comprise sodium chloride, sodium bicarbonate or a mixture of electrolytes or acid components, or any other components known in the art to formulate dialysis fluid. Each reservoir can then be fluidically connected or coupled to an electronically controlled valve (V1, V2, V3, V4). As illustrated, three reservoirs are depicted, although the concept may be generalized to an arbitrary number of reservoirs, thereby enabling three-stream proportioning, four-stream proportioning, five-stream proportioning, six-stream proportioning, etc. Additionally, valve V4 may connect the circuit into a purified water source.

As shown in FIG. 8B, the valve outlets can be connected into a single output line or channel, and this line comprises at least one, but preferably two conductivity sensors (CS1, CS2) in series. This line also connects to the inlet of a pump (Pump1). In some embodiments, the outlet of this pump connects without branching into a mixer, such as a helical or bowtie mixer, and after the mixer is disposed at least one more conductivity sensor (CS3). In this embodiment, the valve array V1, V2, V3, and V4 and Pump1 fully dilutes the concentrates and water into dialysis fluid. The advantages of this is that it requires fewer components and a simpler system; however, the mix ratio of the water to concentrates can be very high (45:1) in which case the control may be challenging.

In another embodiment, the outlet of Pump1 is connected via a tee junction into another line, which feed downstream into the inlet of a separate pump Pump2. In these embodiments, the inlet of Pump2 is split between another purified water source and the outlet of Pump1. In this embodiment, the valve array and Pump1 only partially dilutes the concentrates into the purified water, and thus can use a more favorable mixing ratio. A second dilution from Pump2 then fully dilutes the mixture to the desired concentration for use as dialysis fluid. The principles described below apply to either the embodiment with only Pump1 or the embodiment with Pump1 and Pump2.

In operation, Pump1 is controlled by an electronic controller of the system to operate at a set flow rate. The pressures of the reservoirs CR1, CR2, and CR3 are generally known, either because of hydrostatic head height, or because purified water (from a purified water source that is directed into the reservoirs, not shown but described above) at a known pressure is provided to dissolve the powder in the reservoirs. Then, valves V1 through V4 can be controlled (such as by an electronic controller of the dialysis system, not shown) to sequentially activate, such that one valve, and only one valve, is open at any given time. Preferentially, the sequence is controlled such that after a valve to dispense one of the concentrates is opened and closed (cycled), the valve V4 to the purified water source is cycled, and then a valve to a different concentrate reservoir is cycled. For example, the sequence could be V1 to dispense the concentrate from CR1, then V4 to dispense a bolus of purified water, then V2 to dispense the concentrate from CR2, then V4 to dispense another bolus of purified water, then V3, etc. In this manner, boluses of the different concentrates traveling down the line are physically buffered from one another by a bolus of purified water.

In some embodiments, Pump1 may be a piston pump with a set rotational dispense cycle. In some embodiments, the controller can use a rotation encoder or other device on the pump motor to synchronize at least the start of a valve opening with a certain point in the rotational cycle of the pump. In other embodiments, the pump rotational cycle is much shorter than the time of which any given valve is open, or the pump does not have a flow profile that is significantly correlated with its rotational cycle.

It may be advantageous to be able to vary the concentration of the various components of the dialysis fluid. This can be accomplished by modulating the cycle time of the valves, such that more or less of any given component can be mixed in. It is possible to (for a given dialysis fluid flow rate) hold the speed of Pump1 constant, and then simply vary the timing of the valves to alter the dialysis fluid concentration.

Referring to FIG. 8C, the cycling of the valves gives an illustrative example of the different concentrates being mixed together along with purified water. The fluid stream is the one fed into the inlet of Pump1, and will vary in composition spatially along the flow path. Referring to FIG. 8C, when valve V4 is opened the fluid stream composition comprises water W, then valve V4 is closed and valve V1 is opened, causing the fluid stream composition to comprise only concentrate Cl from reservoir CS1. Next, valve V1 is closed and valve V4 is again opened, causing the fluid stream composition to comprise only water W, and so forth. Once the fluid passes the mixer, it should be substantially mixed homogenously; therefore the conductivity signal at CS3 (in FIG. 8B) should be fairly constant.

However, conductivity sensors CS1 and CS2 are disposed downstream of the valves and upstream of the mixer, and this non-homogenous fluid stream will pass by it prior to being mixed. Purified water has a very low conductivity, as there are few electrolytes to carry electric charge. In contrast, the concentrates have a very high conductivity. Therefore, as this non-homogenous stream passes by the conductivity sensors, a series of peaks and troughs corresponding to various components mixed will be detected, as shown in FIG. 8D. Most conductivity sensors known in the art are unable to distinguish the actual composition of the fluid being measured (for example, the difference between sodium chloride and sodium bicarbonate); however, because of the valve timing described above, flow rate and flow path length between the valve array and the conductivity sensor(s) is known, it is easy to correlate any given peak with the source concentrate.

Because of diffusive effects and other fluid dynamic phenomena, the signal at the conductivity sensor may be more spread out with a rise and fall time, than a perfect step function. Because each bolus of concentrate is buffered by a bolus of purified water, the ability to resolve each peak is improved. The amount of concentrate dispensed can be calculated by quantifying the area under the curve of each peak, or its height, or other parameter. In this manner, it is possible to use a single conductivity sensor to verify that multiple streams are being mixed at the correct ratio.

Another aspect of the disclosure is adaptability to a wide range of flow rates. For example, in stable patients, intermittent hemodialysis (IHD) is delivered with dialysis fluid flow rates from 300-800 mL/min, with treatments lasting 3-4 hours. In more unstable patients, continuous renal replacement therapy (CRRT) is more appropriate, with dialysis fluid flow rates between 10-100 mL/min. In between, slow low-efficiency dialysis (SLED) and nocturnal treatments may operate in between these flow rate ranges. A single machine that can deliver a wide gamut of flow rate ranges has value in terms of therapeutic flexibility. One design challenge that arises is that these flow rates can differ by more than one order of magnitude; therefore the transit time of any given bolus across a conductivity sensor can vary by that same amount. At a high dialysis fluid flow rate, the bolus may transit the conductivity sensor so quickly that the temporal resolution of the sensor is not able to produce an accurate representation of the waveform shape. Conversely, at a very low dialysis fluid flow rate, diffusion of the concentrate across the purified water buffer has more time to occur before contacting the conductivity sensor, and thus may result in the peaks being spread too wide to be resolvable or useful for calculation.

For safety reasons, it is often practiced to employ redundant systems (such as sensors) for critical monitoring and control functionality. In dialysis, the conductivity sensor which monitors the proper mixing of the dialysis fluid may be deployed in at least duplicate, with a primary sensor and a secondary monitor sensor. If the sensors are reading the same signal, and if they disagree by more than a set amount, a safety alarm may be raised. Sometimes, there are differences in the characteristics of the sensors or their communication pathway to reduce the likelihood that a condition that would cause both the sensors to read falsely at the same time.

In the current disclosure and as shown in FIG. 8B, at least two conductivity sensors are disposed along the fluid path upstream of the mixer, CS1 and CS2. One sensor may be optimized to detect peaks at one flow rate, and another sensor may be optimized to detect peaks at a different flow rate. For example, referring to FIG. 8E, the flow path diameter in one of the sensors may be larger (in this example, the flow path diameter of sensor CS2), which increases the effective transit time of a bolus across the sense electrode, increasing the temporal resolution at the cost of spatial resolution. This may be more appropriate for higher flow rates. Alternatively, referring to FIG. 8F, the size of the sense electrode may be smaller in one sensor than the other (in this example, sensor CS2 is smaller than sensor CS1), which achieves the same effect. Depending on the set flow rate, the peaks will resolve better in one sensor than the other. The other sensor may still be functional as a gross detection means and serve as a redundancy check. At other flow rates, the roles of the sensors may be reversed dynamically.

Referring to FIG. 8G, methods may be employed such that the degree of concentrate peaks can overlap and still be calculated. By knowing the valve timing and flow rate, an accurate guess can be made as to where that specific solution should saturate at, thus forming a local maxima. The valve would then be closed to allow for a sharp peak to form in overall conductivity measurement. The conductivity measurement would be multi-modal by utilizing such a timing as each stream would form its own peak depending on its valve window and solution conductivity. To calculate the dose delivered, the global conductivity profile cannot be used as the conductivity sensor is not a selective measurement. However, by utilizing peak deconvolution paired with a peak-guess, based on valve timing, the fundamental profiles can be separated and area's calculated uniquely. Unlike where this is typically employed in spectral analysis where an unknown substance is being analyzed and local peaks measured, this methodology is creating peaks at known time intervals for the algorithm to find. Further the flow rate and conductivity of the local solutions are known thus a well-formed guess can be feed into the peak finding algorithm thus improving accuracy. This can be seen in FIG. 8H. The conductivity labeled as “condo” is a summation of the independent streams. The local streams are inferred based on known parameters about the fluid flow and time correspondence of the peak. The algorithm makes a guess based on the valve on/off time and fluid flow. This method would allow for a quicker valve timing as full profile separation would not be required.

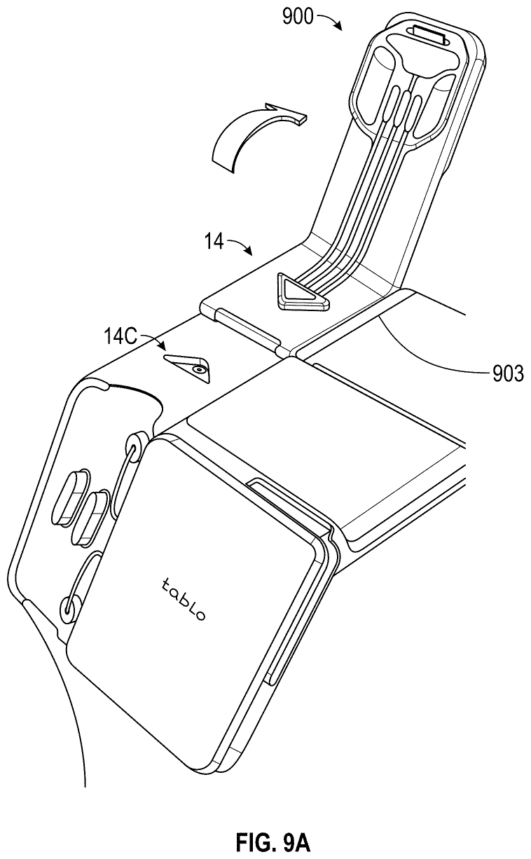

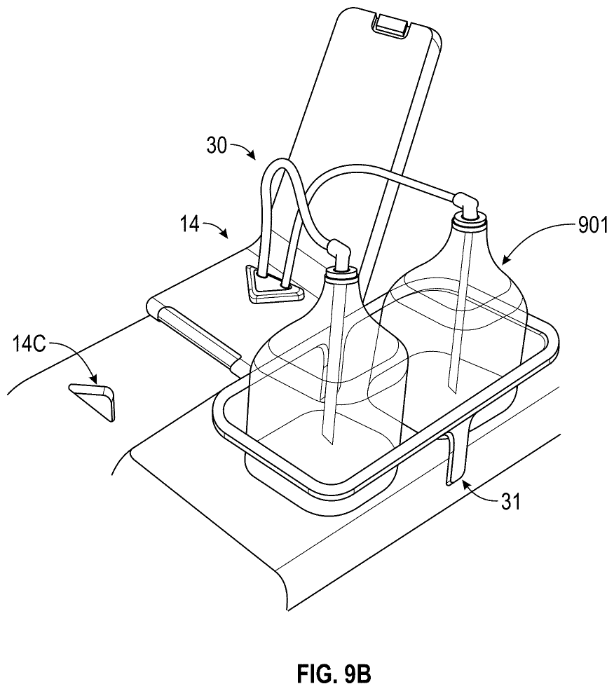

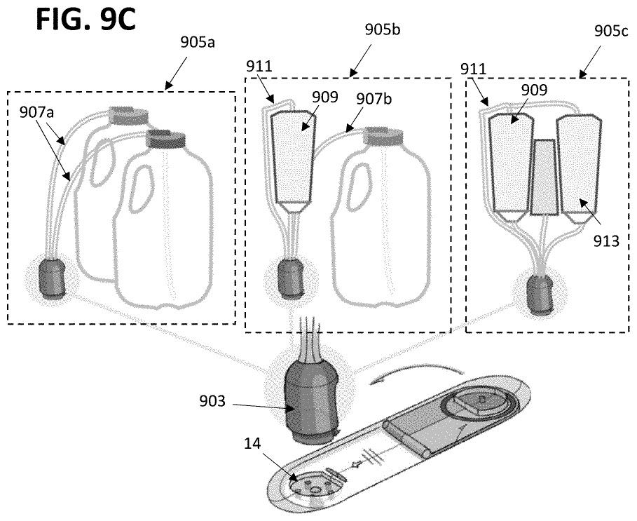

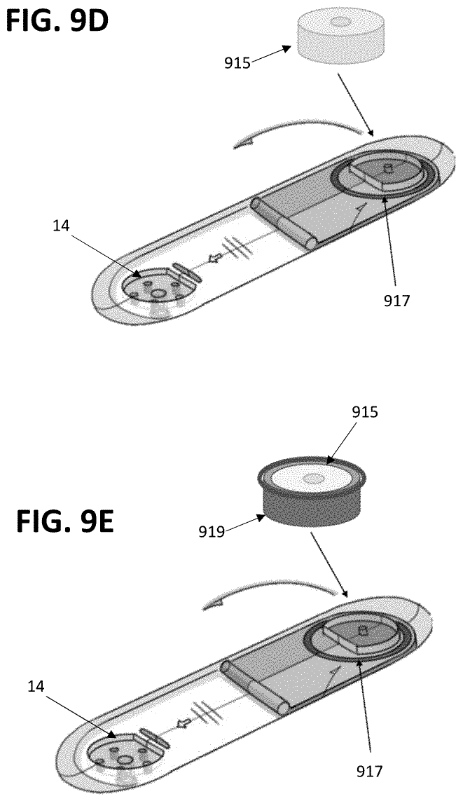

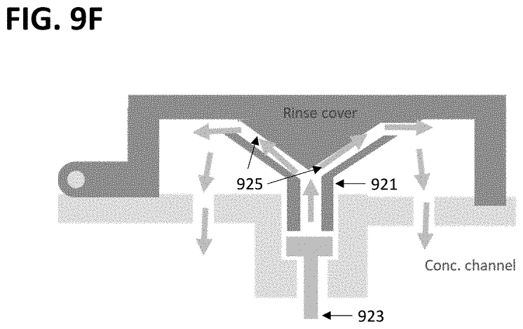

Although this disposable reservoir container described above has many advantages, in some embodiments the dialysis systems described herein can be further configured to generate dialysis from either the disposable container 900, as shown in FIG. 9A, or from normal concentrate jugs 901 intended for use with standard 3-stream proportioning systems, as shown in FIG. 9B. This may be due to operational, cost or availability reasons. Referring to FIG. 9A, as described above, the dialysis system can include a disposable container 900 configured to mount to the dialysis machine via a connector-interface 14. In one embodiment, the connector-interface can be on a hinged cover 903, as shown. In another embodiment, the connector-interface can be on the machine and covered/uncovered by the hinged cover, as shown by reference number 14C.

Referring to FIG. 9B, the dialysis machine can be further configured to accept normal concentrate jugs via connector-interface 14. This can be accommodated with the introduction of an adapter connector 30 in the dialysis system. The two channels of the adapter connector 30 can connect to the connector-interface 14, and are configured to receive acid concentrate and bicarbonate concentrate from the jugs and are able to support flow rates high enough for liquid acid and bicarbonate concentrate, respectively. Adapter connector tubing can thus comprise a connector that mates with the connector-interface 14, forming fluidic connections with those two channels only. Two flexible tubes of the adapter connector 30 can connect those flow channels to acid and bicarbonate jugs. In one embodiment, the adapter connector 30 can include two different tubes with colored markings or identifiers (e.g., red and blue markings, or iconography) to allow the user to identify which connector goes into which jug. Additionally, a rack or support 31 can be mounted to the dialysis machine to hold the jugs in place.

FIG. 9C provides a view of additional details of the connector-interface 14 configured to accept normal concentrate jugs. As shown in FIG. 9C, the connector-interface 14 can be configured to mate with a concentrate/dialysis fluid connector 903 that can interface with a plurality of disposable configurations. In a first configuration 905a, the connector 903 can be fluidly coupled to two streams 907a configured to mate with acid and bicarb concentrate jugs (as described in FIG. 9B). In configuration 905b, the connector 903 can include a built in stream 907b configured to interface with an acid concentrate jug, and can further include a built in disposable powdered bicarb circuit 909. The connector 903 in this configuration can further include a purified water line 911 configured to provide purified water to the disposable powdered bicarb circuit 909. In this configuration, liquid solution of sodium chloride is provided from the jug, and a bicarb solution is produced in real-time by proportioning purified water into the disposable powdered bicarb circuit 909. Finally, the configuration 905c replaces the jug with a disposable powdered sodium chloride circuit 913, and the purified water line 911 is configured to provide purified water to both the disposable powdered bicarb circuit 909 and the disposable powdered sodium chloride circuit 913. The embodiments shown in FIG. 9C show a 4-stream connector/container, but it should be understood that these concepts could be expanded to a 6-stream connector/container as described above in FIG. 4A.

In another embodiment, referring to FIG. 9D, in some embodiments the connector interface 14 described above enables the placement of a disinfecting “pod” 915 into the connector interface 14 after treatment for sanitation and cleaning of the system.

In one embodiment, the pod can be placed into the connector interface 14, or alternatively, in a cover 917 of the system. When the cover is closed, the pod is placed in fluid communication with the machine-side of the system, and the cleaning cycle can be initiated. The system can then be configured to flood the mixing chamber with water to disinfect the system with the pod.

In some embodiments, referring to FIG. 9E, the pod 915 is a mass of powdered citric acid that is contained within a dissolvable container 919, like a dissolvable polymer (PVA). PVAs can be used to package a controlled portion of powdered citric acid. By making use of a PVA container to house the packet of citric acid, this embodiment allows the user to plug in the container and turn on a cleaning cycle. Water then flows through the system to fill the PVA container and dissolve the PVA container, thereby releasing a cleaning solution of a desired concentration from the container. In one embodiment, the cover seal 917 from the embodiment of FIG. 9D is not installed separately in the cover, but instead is a part of the dissolvable container 919 in the embodiment of FIG. 9E.

In yet another embodiment, a stand-alone container can be configured to house a dissolvable packet of powdered cleaning solution held within a captive cage, within the container. The desired strength of the cleaning solution could be controlled by the amount of powdered cleaning solution held within the dissolvable packet. By containing the cleaning solution within a confined packet, use errors of accidental spillage, splashing or leaking are avoided and thus provides a safer implementation for the end user. Unlike conventional dissolvable portion control methods for cleaning which employ a captive container, this solution eliminates the use error of dirt, debris, or unwanted cleaners going into the dialysis machine. The captive container is designed to only fit the proprietary fittings on the system thus eliminating any open hatches where users could place the dissolvable packet. One captive container would serve the use of one cleaning cycle.

In yet another embodiment, in place of a water soluble polymer, a fine mesh strainer could be configured to hold the powdered cleaner within the captive container or a fine cloth like fabric container for the powder, similar to a tea bag. The added step of using a fine mesh strainer would increase manufacturing costs due to the complexities of dealing with an unbound or loose powder.

When the cover described above is closed, water must flow out of the purified water outlet and into the cover to rinse and disinfect the entire area. But the most direct path from the water outlet to the inlet channels is laterally outward, so there may be some difficulty in getting the entire area rinsed and disinfected. Referring to FIG. 9F, the rinse cover can include a finger 921 that is configured to push a poppet valve 923 on the water channel outlet. In some embodiments, the finger can be hollow and can include channels 925 that direct the water flow upwards and outwards towards the periphery of the cover within the rinse cover to create more even circulation for the water to rinse and disinfect “hard-to-reach” areas within the cover.

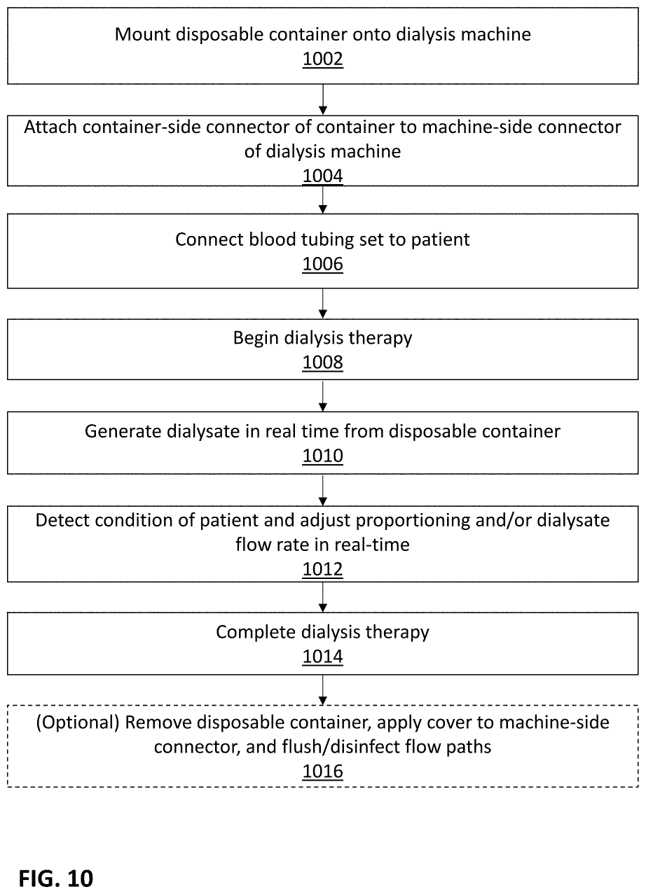

FIG. 10 is a flowchart illustrating one method of providing dialysis therapy to a patient. At step 1002 of FIG. 10, the method can include the step of mounting a disposable container onto a dialysis machine. As described above, the disposable container can include a plurality of compartments configured to hold either liquid or powdered dialysis fluid components. The disposable container can further include flow paths configured to facilitate the delivery of purified water to the container, in addition to flow paths configured to facilitate the delivery of liquid components from the container to the dialysis machine for proportioning. Additionally, the disposable container can include a connector-interface configured to mate or be attached to a corresponding connector-interface on the dialysis machine.

At step 1004 of FIG. 10, the method can include attaching the connector-side connector of the container to the machine-side connector of the dialysis machine. In one embodiment, this connection fluidly connects the flow paths of the container to the flow paths of the dialysis machine. In one implementation, this connection initiates a switch or valve in a purified water line that allows purified water from the dialysis machine to flow into the disposable container.

At step 1006 of FIG. 10, a blood tubing set of the dialysis machine can be connected to a patient, and at step 1008 of FIG. 10, dialysis therapy can begin. As dialysis therapy is underway, at step 1010 of FIG. 10, the dialysis machine can generate dialysis fluid in real-time from the disposable container. This can include delivering purified water from the dialysis machine into the disposable container to mix with powdered dialysis fluid components, in addition to delivering aqueous dialysis fluid components from the disposable container into the dialysis machine. The individual liquid dialysis fluid components can be automatically mixed and proportioned by the dialysis machine to interface with the patient's blood via a dialyzer of the dialysis machine.

At step 1012 of FIG. 10, the dialysis machine can continuously monitor, measure, or sense one or more parameters of the patient or the patient's blood, including electrolyte levels. In some implementations, these measurements can be used to detect a condition of the patient, resulting in the dialysis machine adjusting proportioning and/or dialysis fluid flow rate in real-time based on the condition. At step 1014 of FIG. 10, the dialysis therapy can be completed.

Optionally, at step 1016 of FIG. 10, the method can include removing the disposable container, applying a cover to the machine-side connector of the connector interface, and initiating a flow/disinfect of the flow paths with the dialysis machine. Applying a cover to the machine-side of the connector interface can allow for the flow of fluid/disinfectant through the machine flow paths to remove buildup and contaminants that occur over the course of many treatments. In some embodiments, the cover can be latched or locked into place on the dialysis system when closed/during a disinfection routine. This can be a mechanical latch, or alternatively, can be a magnetic latch that does not have mechanical crevices which could be fouled by concentrates. In one embodiment, a switchable permanent magnet such as from Magswitch™ could be used, which when turned 180 degrees by an electromechanical actuator switches between a very strong permanent magnet to keep the cover in place.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method of providing dialysis therapy, comprising the steps of:
fluidly coupling a disposable container to a dialysis machine with a single connection interface, the disposable container including at least one compartment having a powdered dialysis fluid component therein and at least one compartment having a liquid dialysis fluid component therein;
connecting a blood tubing set of the dialysis machine to a patient;
initiating dialysis therapy;
delivering a purified water into the at least one compartment having the powdered dialysis fluid component;
generating dialysis fluid in real-time from the disposable container; and
initiating dialysis therapy with the dialysis fluid.

2. The method of claim 1, further comprising:
detecting a condition of the patient; and
adjusting proportioning of the dialysis fluid in real-time.

3. The method of claim 1, wherein mounting the disposable container further comprises attaching a connector-side interface of the disposable container to a corresponding machine-side interface on the dialysis machine.

4. The method of claim 2, wherein detecting a condition further comprises measuring at least one parameter of the patient's health or the patient's blood.

5. The method of claim 2, wherein the condition comprises a state of electrolytes in the patient's blood.

6. The method of claim 2, wherein adjusting proportioning further comprises increasing a concentration of at least one dialysis fluid component from the disposable container.

7. The method of claim 6, wherein increasing a concentration of at least one dialysis fluid component further comprises increasing a proportion of fluid from the at least one compartment having the powdered dialysis fluid component.

8. The method of claim 6, wherein increasing a concentration of at least one dialysis fluid component further comprises increasing a proportion of fluid from the at least one compartment having the liquid dialysis fluid component.

9. The method of claim 1, further comprising:
decoupling and removing the disposable container from the dialysis machine;
applying a cover to a machine-side connector of the single connection interface; and
initiating a disinfect cycle through one or more flow paths of the dialysis machine and the machine-side connector.

10. A disposable container configured to facilitate real-time dialysis fluid production, comprising:
at least one powder compartment configured to hold a powdered dialysis fluid component;
at least one liquid compartment configured to hold a liquid dialysis fluid component;
a connector interface configured to mate with a dialysis machine;
at least one inlet flow path configured to deliver purified water from the dialysis machine through the connector interface to the at least one powder compartment; and
a plurality of outlet flow paths configured to deliver dialysis fluid components from the at least one powder compartment and the at least one liquid compartment to the connector interface and the dialysis machine.

11. The disposable container of claim 10, wherein the at least one powder compartment comprises a NaCl powder compartment and a NaHCO₃ powder compartment.

12. The disposable container of claim 10, wherein the at least one liquid compartment comprises a C₆H₈O₇ liquid compartment, a C₆H₁₂O₆ liquid compartment, and a MgCl₂ liquid compartment.

13. The disposable container of claim 10, further comprising a diffuser/filter disposed in the outlet flow paths between the at least one powder compartment and the connector interface.

14. The disposable container of claim 10, wherein the connector interface further comprises a container-side connector interface configured to mate with a corresponding machine-side connector interface.

15. The disposable container of claim 14, wherein the connector interface comprises at least one inlet flow channel and a plurality of outlet flow channels.

16. The disposable container of claim 15, wherein the at least one inlet flow channel is fluidly coupled to a source of purified water.

17. The disposable container of claim 16, wherein the at least one inlet flow channel is configured to deliver purified water to the at least one powder compartment.

18. The disposable container of claim 17, wherein the machine-side connector interface further comprises a valve disposed in the at least one inlet flow channel, wherein the valve is configured to open when the container-side connector interface is connected to the machine-side connector interface.

19. The disposable container of claim 15, wherein each of the plurality of outlet flow channels comprises a pump configured to deliver a dialysis fluid from the at least one powder compartment and the at least one liquid compartment to the dialysis machine.

20. The disposable container of claim 15, wherein each of the plurality of outlet flow channels comprises a flow sensor configured to measure a flow rate of dialysis fluid from the at least one powder compartment and the at least one liquid compartment to the dialysis machine.

21. The disposable container of claim 10, wherein the at least one powder compartment and the at least one liquid compartment are large enough to generate enough dialysis fluid to support multiple dialysis treatments.

22. A dialysis machine, comprising:
a connector interface disposed on or in the dialysis machine, the connector interface being configured to be coupled with a container including one or more sources of dialysis fluid, the connector interface being coupled to at least one purified water flow channel and a plurality of flow channels configured to receive the one or more sources of dialysis fluid when the container is coupled to the connector interface; and
a rinsing cover disposed on the dialysis machine, the rinsing cover being configured to be moved into a rinsing configuration in which the rinsing cover forms a fluidic seal with the connector interface, wherein in the rinsing configuration purified water flows from the at least one purified water flow channel into a volume defined by the rinsing cover and the connector interface and further flows into the plurality of flow channels.

23. The dialysis machine of claim 22, wherein in the rinsing configuration, a valve opening member of the rinsing cover is configured to open a valve in the at least one purified water flow channel.

24. The dialysis machine of claim 22, wherein the volume is further configured to receive a disinfecting pod.

25. The dialysis machine of claim 22, wherein the rinsing cover further comprises one or more fluid channels configured to direct the purified water towards a periphery of the rinsing cover.
